(19) United States
(12) Patent Application Publication
(10) Pub. No.: US 2022/0009900 A1
(43) Pub. Date: Jan. 13, 2022

(54) N-TRIFLUORMETHYLCARBONYL COMPOUNDS AND METHODS FOR THEIR SYNTHESIS

(71) Applicant: RHEINISCH-WESTFÄLISCHE TECHNISCHE HOCHSCHULE (RWTH) AACHEN, Aachen (DE)

(72) Inventors: Franziska SCHOENEBECK, Köln (DE); Thomas SCATTOLIN, St. Georges les Baillargeaux (FR)

(73) Assignee: RHEINISCH-WESTFÄLISCHE TECHNISCHE HOCHSCHULE (RWTH) AACHEN, Aachen (DE)

(21) Appl. No.: 17/295,789

(22) PCT Filed: Nov. 19, 2019
(86) PCT No.: PCT/EP2019/081850

§ 371 Date: May 20, 2021

(30) Foreign Application Priority Data
Nov. 23, 2018 (DE) 10 2018 129 597.9

(51) Int. Cl. C07D 333/38; C07C 269/02; C07C 273/18

(52) U.S. Cl. CPC C07D 333/38; C07C 273/1809; C07C 269/02

(57) ABSTRACT

The present invention relates to the synthesis of N-trifluoromethylcarbonyl compounds starting from isothiocyanates, which can be converted into N-trifluoromethylcarbamoyl fluorides or similar compounds, which later on can be derivatized further to amides, ureas, carbamates, thiocarbamates and selenocarbamates.

The present invention relates to the field of N-trifluoromethyl compounds, especially N-trifluoromethylcarbonyl compounds.

The amide motif is of central importance, impacting all sciences, life, medicine and modern society. It is a preferred building block of nature and a key unit that controls stability, 3D-structure and hence function of proteins or peptides, and consequently also biological processes or related applications, such as catalysis.

The closely related compound families, e.g. carbamates and ureas, are similarly influential, finding applications as insecticides, in polymers (foams, elastomers, polyurethanes), as preservatives, cosmetics and in medical treatment as, for example, chemotherapy and HIV agents.

Fluorinated compounds play an eminent role, especially but not limited to the pharmaceutical field due to the steric similarity but different chemical/physical behaviour between fluorine and hydrogen and thus methods for the specific synthesis of fluorinated compounds are subject of immense research.

Due to the importance of amides and related compounds, there is a constant need for methods for the synthesis of fluorinated amides and related compounds, especially compounds which comprise an N-CF₃ motif. Additionally, there is a significant demand for novel compounds having such an N-CF₃ motif.

This object is met by the method of claim 1 of the present invention. Accordingly, a method for the synthesis of N-trifluoromethylcarbonyl compounds having the structure R—N(CF₃)CO—LG, whereby R is an organic moiety from isothiocyanates R-NCS and LG is a leaving group is provided, comprising the step a)
a) contacting the isothiocyanate R—NCS with AgF and an electrophilic C₁-compound where the carbon is in the formal oxidation state (+IV)

Surprisingly, it has been found that by doing so for many isothiocyanates a conversion into N-trifluoromethylcarbonyl compounds can be observed. For most applications within the present invention, at least one of the following advantages could be observed:
The reaction usually proceeds smoothly and straightforwardly.
The reaction can be performed at room temperature or slightly elevated temperatures.
The reaction avoids toxic reactants such as HgF2 which have been used before.
A wide range of isothiocyanate reactants can be used, including reactants which have a complex structure, which is usually not affected in the course of the reaction.
Isothiocyanates are substances which are easily obtainable, thus the reaction can be used handily in many fields of organic chemistry.
The reaction usually occurs with retention of configuration.
A wide variety of functional groups are tolerated.

The term “organic moiety” especially means and/or includes a structure whereby a carbon atom is adjacent to the N(CF₃)CO-unit and bound to that unit with a single bond.

The term “leaving group” especially means and/or includes any chemical moiety which is able to depart with a pair of electrons in heterolytic bond cleavage. Especially preferred leaving groups are halogens, here especially preferred fluoride and/or chloride, sulfonic esters, especially tosylates, heterocyclic compounds, especially imidazoles. Especially preferred are fluoride and chloride.

The term “electrophilic C₁-compound” especially means and/or includes a compound of the chemical structure LG¹-13 CO-LG² whereby LG¹ and LG² are independently from each other leaving groups, including leaving groups as above. However, also preferred in this context are leaving groups of the structure -OCHal₃, whereby Hal is halogen, especially fluoride and/or chloride. The resulting compounds, e.g. Hal₃C—O(CO)O-CHal₃ have the advantage that they in the course of the reaction will form two compounds Hal-CO-Hal (as known e.g. from triphosgene, the compound with Hal=C1) which themselves are electrophilic C₁-compounds. Similarly a compound where only LG is -OCHal₃ will form additionally (only) one compound Hal-CO-Hal (e.g. known from diphosgene, Cl—CO—OCl).

AgF can be used either directly or in its components, i.e. via a cationic silver component (e.g. AgNO₃, AgOTf) and nucleophilic fluoride (e.g. KF, NaF).

It should be noted that the isothiocyanate R-NCS and/or the electrophilic C₁-compound and/or the AgF may be formed in situ during the course of the reaction. Therefore it is a preferred embodiment of the present invention that the method comprises another step a0) which is performed before step a)

a0) Formation of the isothiocyanate R—NCS and/or the electrophilic C₁-compound and/or the AgF

According to a preferred embodiment of the present invention, AgOCF₃ is used to directly transform R—NCS to N—CF₃ carbamoyl fluorides. The AgOCF₃ generates AgF and COF₂ in situ.

Other OCF₃-containing compounds can also be used as COF₂ source.

It is especially advantageous that the molar ratio of AgOCF₃ to isothiocyanate is ≥1.0, preferably ≥1.1 to ≤3, more preferred ≥1.5 to ≤2,5, most preferred ≥1.8 to ≥2.

The AgOCF₃ itself can be provided in substance or itself be generated in situ (either in a separate step or during step a0).

According to a further preferred embodiment of the present invention, AgF is formed in situ by cross-reaction of a silver salt and a nucleophilic fluoride compound, whereby suitable silver salt precursors include AgOTf, AgNO₃, Ag halides (other than fluoride) and nucleophilic fluoride compounds include tetraalkylammonium fluorides and alkali fluorides.

The fluoride can also be formed in situ from an organic compound that contains fluoride by suitable chemical transformation, e.g. by a nucleophilic aromatic substitution reaction of a suitable aryl fluoride with a nucleophile or by displacement from alternative fluorine containing compounds, such as, for example R—SO₂—F.

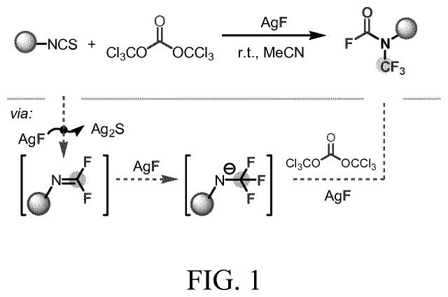

Without being bound to any chemical theory, the inventors believe that when an excess of AgF is used (cf. the further description) then in most applications of the present invention a conversion of the isothiocyanate to an N-trifluoromethylcarbamoyl fluoride occurs according to the mechanism shown in FIG. 1 of this application for a special embodiment of the present invention:

Accordingly, the sulfur of the isothiocyanate is replaced by two fluorides via an exchange reaction with the AgF, which subsequently reacts to Ag₂S. Thus a compound of the structure R—N═CF₂ is formed. Additional fluoride forms the CF₃-unit following a nucleophilic attack of the nitrogen to the carbon of the electrophilic C₁-compound. If fluoride is used in excess, then another substitution leads to the N-trifluoromethylcarbamoyl fluoride.

According to a preferred embodiment of the present invention, in step a) the molar ratio of effective electrophilic C₁ compound to isothiocyanate is ≥1.0 to ≤2, preferably ≥1.2 to ≤1.5. The term “effective” means that if in the course of the reaction the additional electrophilic C₁ compound is formed then this is taken into account, e.g. when triphosgene is used as electrophilic C₁ compound then the amount of effective electrophilic C₁ compound is three times the amount of triphosgene. When diphosgene Cl—CO—OCl₃ is used, the effective electrophilic C₁ compound is twice the amount of diphosgene.

According to a preferred embodiment step a) is carried out in the presence of one or more additional fluoride compounds, which are preferably used in a molar ratio of ≥1 (compared to the isothiocyanate). Preferred fluorine compounds are alkali and earth alkali fluorides, especially LiF, NaF, KF, RbF, CsF, CaF₂, MgF₂, fluorides from quaternary amines, especially TMAF and TBAF (NMe₄F, NBu₄F) and other metal fluorides, especially CuF₂. Preferably the fluorine compound is used in a molar ratio of this fluorine compound to the isothiocyanate in ≥1, still preferred ≥2 and most preferred ≥3 to ≤4.

According to a preferred embodiment of the present invention, in step a) the molar ratio of AgF to isothiocyanate in step a) is ≥4, preferably ≥5 to ≤8. When doing so usually the N-trifluoromethylcarbamoyl fluoride is formed, as described above. In many cases the yields are so high that AgF can be used as sole fluorine compound which is therefore an embodiment of the present invention.

However, according to an alternative preferred embodiment of the present invention, in step a) the molar ratio of AgF to isothiocyanate is ≥3 to ≤4. When doing so then—in case no further fluoride compound is present, which is, however, a preferred embodiment of the invention—in many applications in the product R—N(CF₃)CO—LG the moiety LG will be the LG of the electrophilic C₁-compound or a mixture of this compound with N-trifluoromethylcarbamoyl fluoride can be observed as reaction products. It goes without saying that in case the leaving group of the electrophilic C₁-compound is —OCHal₃ then of course LG will be Hal.

Yet another to an alternative preferred embodiment of the present invention in step a) the molar ratio of AgF to isothiocyanate is ≥2 and another fluorine compound is used in a molar ratio of ≥1 (compared to the isothiocyanate). Also here, depending on the fluoride concentration, in many applications in the product R—N(CF₃)CO-LG the moiety LG will be the LG of the electrophilic C₁-compound or a mixture of this compound with N-trifluoromethylcarbamoyl fluoride can be observed as reaction products.

Preferably step a) is carried out in an organic nonpolar or dipolar aprotic solvent. Especially preferred are nitrile-based solvents, especially acetonitrile, benzonitrile, propionitrile, ether-based solvents, especially glyme, diglyme, triglyme, MeTHF, Et₂O, THF, dioxane, NMP, DCM, toluene, THF with acetonitrile being most preferred.

Preferably step a) is carried out at a temperature of ≥0° C. and ≤70° C., more preferred >20° C. and ≤30° C. In case that AgOCF₃ is used as an AgF and/or COF₂ precursor, preferably the method is carried out at a temperature of ≥30° C. and ≤70° C., more preferred ≥40° C. and ≤50° C.

The object of the present invention is furthermore met by the method of claim 8 of the present invention. Accordingly a method for the synthesis of N-trifluoromethyl amides is provided comprising the step of contacting a compound R—N(CF₃)CO-LG, with R and LG as defined above, with a organometallic compound.

Surprisingly it has been shown that by doing so, N-trifluoromethyl amides can be obtained in excellent yields for many applications. For most applications within the present invention, at least one of the following advantages could be observed:
The reaction usually proceeds smoothly and straightforwardly.
The reaction can be performed at room temperature or slightly elevated temperatures.
The reaction usually occurs with retention of configuration.
The reaction can occur without temperature control.
Reaction can occur without the need for careful reaction control [addition of the reactant].

Preferred metal-organic compounds are organomagnesium compounds (Grignard Reagents), organolithium compounds and organozinc compounds.

Preferably the method is carried out in an organic nonpolar or dipolar aprotic solvent. Especially preferred are DCM, Et₂O, toluene, THF with toluene being most preferred.

Preferably the method is carried out at a temperature of ≥−100° C. and ≤25° C., more preferred ≥0° C. and ≤25° C.

The object of the present invention is furthermore met by the method of claim 10 of the present invention. Accordingly a method for the synthesis of N-trifluoromethyl ureas, N-trifluoromethyl carbamates, N-trifluoromethyl thiocarbamates and/or N-trifluoromethyl selenocarbamates is provided, comprising the step of reacting a compound R—N(CF₃)CO-LG, whereby R is an organic moiety and LG is a leaving group, with an amine, alcohol, thiol and selenol.

Surprisingly it has been shown that by doing so these compounds can be obtained in excellent yields for many applications. For most applications within the present invention, at least one of the following advantages could be observed:
The reaction usually proceeds smoothly and straightforwardly.
The reaction can be performed at room temperature or slightly elevated temperatures.
The reaction usually occurs with retention of configuration.
The reaction can occur with the neutral nucleophile or the deprotonated nucleophile.
The reaction proceeds in a highly selective manner.

N-trifluoromethyl carbamates and/or N-trifluoromethyl thiocarbamates which are both very suitable compounds, especially in the pharma sector, are so far unknown, same as selenocarbamates derivatives. Many of them have, however, shown to have a greater stability than their N-methyl-counterparts. Therefore, the present invention also relates to compounds of the structure R—N(CF₃)—CO—X—R′ with R and R′ being organic moieties and X being S, O or Se.

Preferably the method is carried out in an organic nonpolar or dipolar aprotic solvent. Especially preferred are DCM, Et₂O, toluene, THF with DCM and THF being most preferred.

Preferably the method is carried out at a temperature of ≥0° C. and ≤50° C., more preferred ≥20° C. and ≤30° C.

Preferably the method is carried out using a base in an amount of ≥1 and ≤5 equivalents, relative to the reactant R—N(CF₃)CO-LG. Preferred bases are inorganic bases such as, carbonate, bicarbonate and phosphate, such as cesium carbonate, sodium bicarbonate and potassium phosphate, tertiary amines, especially trialkylamines such as triethylamine, Hünig's base (N,N-diisopropylethylamine), N-methylmorpholine, pyridine, 1,8-diazabicyclo[5.4.0]undec-7-ene (DBU), 1,5-diazabicyclo[4.3.0]non-5-ene (DBN), 1,4-diazabicyclo[2.2.2]octane (DABCO) or mixtures thereof

Preferably the method is carried out using ≥0.1 and ≤0.5 equivalents (relative to the reactant R—N(CF₃)CO-LG) of a nucleophilic catalyst selected out of the group comprising pyridine-based catalysts, especially pyridine and DMAP and five-membered N-heterocyclic compounds, especially imidazole. This has shown to furthermore increase yields in many embodiments of the invention.

It goes without saying that in the two methods described before the reactant R—N(CF₃)CO-LG is preferably synthesized as described before, which is insofar a preferred embodiment of the invention. Therefore the present invention relates to a method comprising the steps:
a) contacting an isothiocyanate R-NCS with AgF and an electrophilic C₁-compound where the carbon is in the formal oxidation state (+IV) to obtain a compound R—N(CF₃)CO-LG
b) contacting a compound R—N(CF₃)CO-LG with an organometallic compound, amine, alcohol, thiol and/or selenol.

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

Additional details, characteristics and advantages of the object of the invention are disclosed in the sub-claims and the following description of the respective examples—which in an exemplary fashion--show preferred embodiments according to the invention. Such embodiments do not necessarily represent the full scope of the invention, however, and reference is made therefore to the claims and herein for interpreting the scope of the invention. It is to be understood that both the foregoing general description and the following detailed description are exemplary and explanatory only and are intended to provide further explanation of the present invention as claimed.

General Procedure for the Synthesis of N-trifluoromethylcarbamoyl Fluorides:

A 20 mL vial was charged with the isothiocyanate (2 mmol, 1 equiv.), silver fluoride (10 mmol, 5 equiv.) and triphosgene (237 mg, 0.8 mmol, 0.4 equiv.). Acetonitrile (10 mL) was added quickly and the vial was sealed (if the isothiocyanate was a liquid or an oil it was added as a solution in the solvent). The mixture was stirred at room temperature. Afterwards the crude mixture was added at once to Et₂O (40 mL) and stirred for 10 minutes. The solid was filtered through celite and the solvents were then evaporated. The obtained crude material was dissolved in Et₂O and filtered on a pad of celite again to remove the last traces of salt byproducts. The N-trifluoromethylcarbamoyl fluoride was then obtained in a technical grade purity ranging from 90 to 99%.

Using this general procedure, the following compounds 1 to 30 were synthesized:

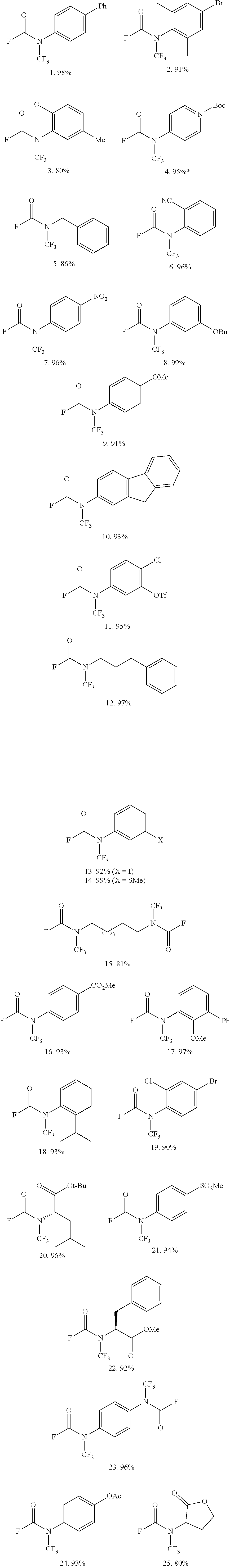

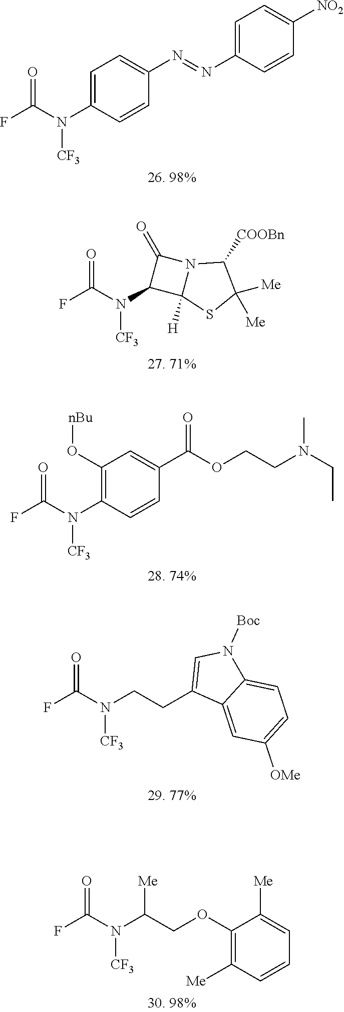

Exemplarily the Synthesis of Compound 1 is Described in More Detail:

[1,1′-Biphenyf]-4-yl-N-trifluoromethylcarbamoyl fluoride 1: The title compound was obtained as a white solid after 15 h in 98% yield (555 mg) using 4-isothiocyanato-1,1′-biphenyl (423 mg) following the general procedure for N-trifluoromethylcarbamoyl fluorides. M.p.: 94-96° C. ¹H NMR (400 MHz, CDCl₃) δ 7.71-7.64 (m, 2H), 7.61-7.54 (m, 2H), 7.50-7.43 (m, 2H), 7.43-7.34 (m, 3H). ¹⁹F NMR (376 MHz, CDCl₃) δ-2.6 (brs, 1F), −56.2 (brs, 3F). ¹³C NMR (151 MHz, CDCl₃) δ143.6, 142.1 (d, J=300.8 Hz), 139.4, 132.0, 129.0, 128.6, 128.6, 128.2, 127.3, 119.2 (q, J=264.8 Hz). MS (70eV, EI): m/z (%): 283 (100) [M+], 172 (19), 69 (5). HRMS (EI) calculated for C₁₄H₉ONF₃: 264.06308 [M-F]⁺, Found: 264.06302.

General Procedure for the Synthesis of Amides:

A 4 mL vial was charged with the N-trifluoromethylcarbamoyl fluoride (0.2 mmol, 1 equiv.), toluene (1.5 mL) and placed into a water bath at room temperature. The corresponding Grignard reagent (0.24 mmol, 1.2 equiv.) was subsequently added to the solution. The reaction mixture was stirred for 10 minutes at room temperature. Saturated aqueous ammonium chloride solution (1.5 mL) was then added. The phases were separated and the aqueous phase was further extracted with EtOAc (2×), dried over MgSO₄ and concentrated under reduced pressure. The obtained crude material was then purified by column chromatography.

Using this general procedure the compounds 31 to 44 were synthesized:

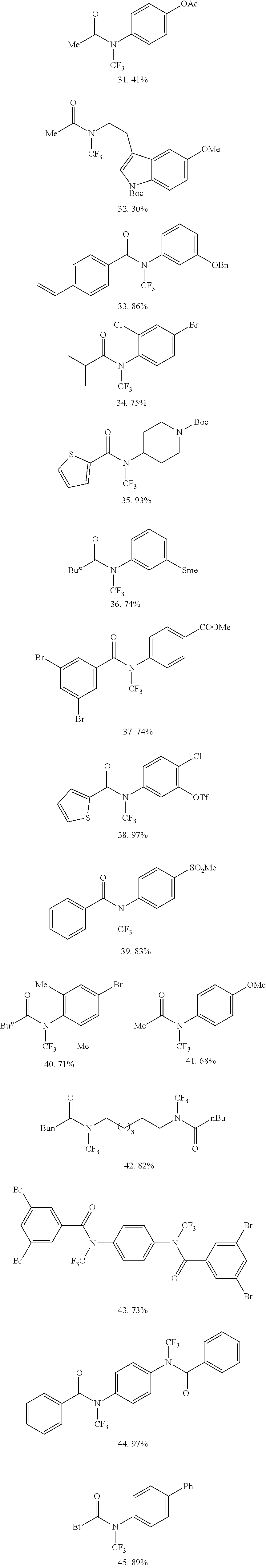

Exemplarily the Synthesis of Compound 38 is Described in Detail:

2-Chloro-5-(N-(trifluoromethyl) thiophene-2-carboxamido)phenyl trifluoromethanesulfonate 38 : The title compound was obtained as a colorless oil in 97% yield (88 mg) using 2-chloro-5-((fluorocarbonyl)(trifluoromethyl)amino)phenyl trifluoromethanesulfonate 11 (78 mg) with 2-thienylmagnesium bromide (240 μL, 1.0M in THF) after column chromatography on silica gel with EtOAc/hexane (5/95, R_{f}=0.20). ¹H NMR (400 MHz, CDCl₃) δ 7.60 (dd, J=8.6, 1.0 Hz, 1H), 7.50 (dd, J=5.0, 1.0 Hz, 1H), 7.42 (d, J=2.4 Hz, 1H), 7.35 (dd, J=8.6, 2.4 Hz, 1H), 7.25-7.23 (m, 1H), 6.92 (ddd, J=5.0, 3.9, 1.0 Hz, 1H). ¹⁹F NMR (376 MHz, CDCl₃) δ-56.5 (s, 3F), −73.2 (s, 3F). ¹³C NMR (151 MHz, CDCl₃) δ 161.5, 145.8, 135.5, 135.3, 134.7, 134.1, 132.1, 131.2, 129.9, 127.5, 125.7, 119.8 (q, J=265.8 Hz), 118.5 (q, J=320.9 Hz). HRMS (ESI) calculated for C₁₃H₆O₄N³⁵ClF₆Na³²S₂: 475.92232 [M+Na]⁺, Found: 475.92151.

General Procedure for the Synthesis of Ureas:

A 4 mL vial was charged with the N-trifluoromethylcarbamoyl fluoride (0.2 mmol, 1 equiv.) and CH₂Cl₂ (1.5 mL). The corresponding amine (0.24 mmol, 1.2 equiv.), Hunig's base (42 μL, 0.24 mmol, 1.2 equiv.) and DMAP (2 mg, 0.02 mmol, 0.1 equiv.) were subsequently added to the solution. The reaction mixture was stirred for 15 h at room temperature. Hexane (1 mL) was then added and the reaction mixture was filtered through a pad of celite before evaporation. The obtained crude material was then purified by column chromatography.

Stability Investigation of Compound 45

To challenge the stability of N—CF₃ amides, we subjected amide 45 to a solution of 0.5 M HCl and also 0.5 M NaOH at 50° C. for 15 h (in 50:50 THF/H₂O) and observed essentially no decomposition, whereas the corresponding N—Me and N—H analogues showed decomposition. This suggests that many N-trifluoromethyl amides are more stable than their ‘conventional’ amide counterparts.

General Procedure for the Synthesis of Carbamates:

A 4 mL vial was charged with the N-trifluoromethylcarbamoyl fluoride (0.2 mmol, 1 equiv.) and CH₂Cl₂ (1.5 mL). The corresponding alcohol (0.24 mmol, 1.2 equiv.), Hunig's base (42 μL, 0.24 mmol, 1.2 equiv.) and DMAP (2 mg, 0.02 mmol, 0.1 equiv.) were subsequently added to the solution. The reaction mixture was stirred for 15 h at room temperature. Hexane (1 mL) was then added and the reaction mixture was filtered through a pad of celite before evaporation. The obtained crude material was then purified by column chromatography.

General Procedure for the Synthesis of Thiocarbamates and Selenocarbamates:

A 4 mL vial was charged with the N-trifluoromethylcarbamoyl fluoride (0.2 mmol, 1 equiv.) and THF (1.5 mL). The corresponding sodium thiolate or selenolate (0.24 mmol, 1.2 equiv.) was subsequently added to the solution. The reaction mixture was stirred for 1 h at room temperature. Hexane (1 mL) was then added and the reaction mixture was filtered through a pad of celite before evaporation. The obtained crude material was then purified by column chromatography.

Using the above general methods, the following compounds 46 to 74 were synthesized:

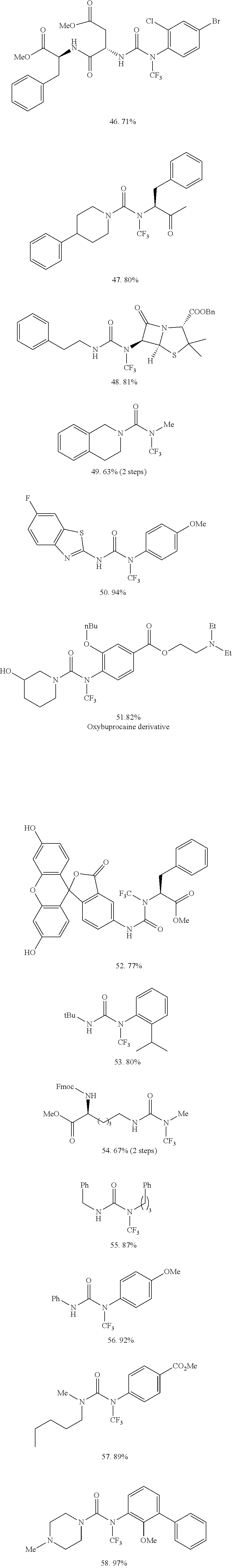

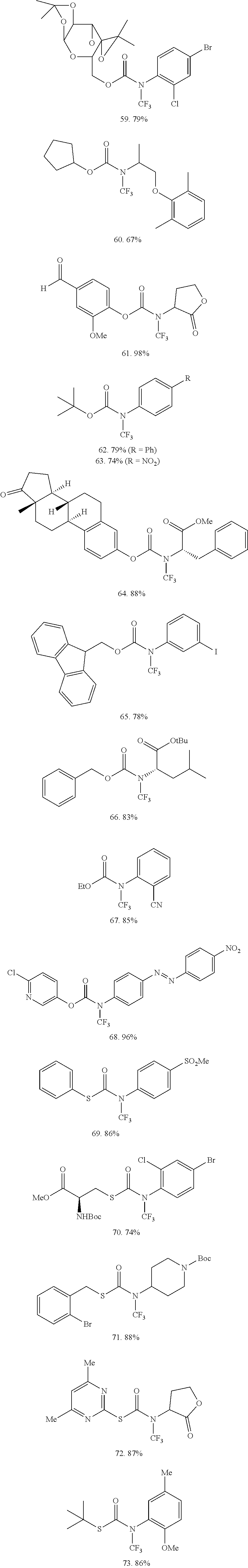

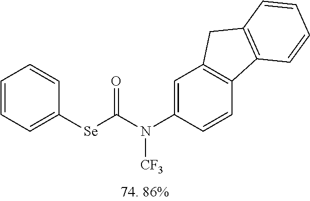 [SMILES: O=C([Se]c1ccccc1)N(c1ccc2c(c1)Cc1ccccc1-2)C(F)(F)F]

Exemplarily the Synthesis of Compound 46 is Described in Greater Detail:

Methyl (S)-3-(3-(4-bromo-2-chlorophenyl) -3-(trifluoromethyl)ureido)-4-(((S)-1-methoxy-1-oxo-3-phenylpropan-2-yl)amino)-4-oxobutanoate 46 : The title compound was obtained as a colorless oil in 71% yield (87 mg) using (4-bromo-2-chlorophenyl)-N-trifluoromethylcarbamoyl fluoride 19 (64 mg) with methyl (S)-3-amino-4-(((S)-1-methoxy- 1-oxo-3-phenylpropan-2-yl)amino)-4-oxobutanoate hydrochloride [aspartame-OMe.HCl] (83 mg) in presence of N,N diisopropylethylamine [Hunig's base] (77 μL, 0.44 mmol, 2.2 equiv.) after column chromatography on silica gel with EtOAc/hexane (40/60, R_{f}=0.38). [α]_{D}=+40.2 (c=0.600, CHCl₃, 26° C.). ¹H NMR (400 MHz, CDCl₃) δ 7.69 (d, J=2.2 Hz, 1H), 7.48 (d, J=8.5 Hz, 1H), 7.29-7.25 (m, 3H), 7.17 (d, J=8.5 Hz, 1H), 7.07 (dd, J=6.6, 2.9 Hz, 2H), 6.93 (d, J=7.6 Hz, 1H), 6.04 (s, 1H), 4.80-4.59 (m, 2H), 3.69 (s, 3H), 3.62 (s, 3H), 3.10 (dd, J=14.0, 5.3 Hz, 1H), 3.04-2.95 (m, 2H), 2.55 (dd, J=17.3, 7.0 Hz, 1H). ¹⁹F NMR (376 MHz, CDCl₃) δ-53.6. ¹³C NMR (151 MHz, CDCl₃) δ 172.6, 171.2, 169.5, 151.4, 136.0, 135.5, 133.9, 132.7, 132.0, 130.9, 129.2, 128.6, 127.1, 125.1, 119.6 (q, J=262.5 Hz), 53.7, 52.4, 52.2, 49.9, 37.5, 35.5. HRMS (ESI) calculated for C₂₃H₂₂⁷⁹Br³⁵ClF₃N₃NaO₆: 630.02248 [M+Na]⁺, Found: 630.02124.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. The invention's scope is defined in the following claims and the equivalents thereto. Furthermore, reference signs used in the description and claims do not limit the scope of the invention as claimed.

1. A method for the synthesis of N-trifluoromcthylcarbonyl compounds having the structure R—N(CF₃)CO—LG, whereby R is an organic moiety and LG is a leaving group, from isothiocyanates R—NCS, comprising the step of
a) contacting the isothiocyanate R—NCS with AgF and an electrophilic C₁-compound where the carbon is in the formal oxidation state (+IV).

2. The method according to claim 1, whereby the electrophilic C₁-compound comprises a compound of the chemical structure LG¹—CO-LG² whereby LG¹ and LG² are independently from each other leaving groups.

3. The method according to claim 1, whereby the method is carried out in the presence of one or more additional fluoride compounds.

4. The method according to claim 1, whereby the molar ratio of AgF to isothiocyanate is ≥4.

5. The method according to claim 1, whereby the molar ratio of effective electrophilic C₁ compound to isothiocyanate is ≥1.0 to ≤2.

6. The method according to claim 1, whereby the isothiocyanate R-NCS and/or the electrophilic C₁-compound and/or the AgF are formed in situ during the course of the reaction.

7. The method according to claim 1, whereby AgOCF₃ is used as a precursor for AgF and the electrophilic C₁-compound.

8. A method for the synthesis of N-trifluoromcthyl amides comprising the step of contacting a compound R—N(CF₃)CO-LG, whereby R is an organic moiety and LG is a leaving group, with an organometallic compound.

9. The method according to claim 8, whereby the organometallic compound is an organomagnesium compound, organolithium compound and/or an organozinc compound.

10. A method for the synthesis of N-trifluoromcthyl ureas, N-trifluoromcthyl carbamates, N-trifluoromcthyl thiocarbamates and/or N-trifluoromcthyl selenocarbamates comprising the step of contacting a compound R—N(CF₃)CO— LG, whereby R is an organic moeity and LG is a leaving group, with an amine, alcohol, thiol or selenol.

11. The method according to claim 8, whereby the compound R— N(CF₃)CO-LG is prepared from isothiocyanates R—NCS, by a method comprising the step of
contacting the isothiocyanate R—NCS with AgF and an electrophilic C₁-compound where the carbon is in the formal oxidation state (+IV).

12. Compounds of the structure R—N(CF₃)—CO—X—R′ with R and R′ being organic moieties and X being S, O or Se.

13. The method according to claim 10, whereby the compound R—N(CF₃)CO-LG is prepared from isothiocyanates R-NCS by a method comprising the step of
contacting the isothiocyanate R—NCS with AgF and an electrophilic C₁-compound where the carbon is in the formal oxidation state (+IV).
